(19) United States
(12) Patent Application Publication
(10) Pub. No.: US 2009/0148879 A1
(43) Pub. Date: Jun. 11, 2009

(54) Stabilizing Compositions, Methods and Kits for Chemiluminescent Assays

(76) Inventors: Phillip G. Mattingly, Third Lake, IL (US); Maciej B. Adamczyk, Gurnee, IL (US); Roy Jeffrey Brashear, Mundelein, IL (US)

Correspondence Address: Robert DeBerardine;D-377/AP6A-1, Abbott Laboratories, 100 Abbott Park Road, Abbott Park, IL, 60064-6008, US

(21) Appl. No.: 11/953,245

(22) Filed: Dec. 10, 2007

(51) Int. Cl. C12Q 1/28; G01N 33/48; C12Q 1/30

(52) U.S. Cl. 435/27; 435/40.5; 435/28

(57) ABSTRACT

The present invention relates to stabilizing compositions, methods and kits for chemiluminescent assays.

### RELATED APPLICATION INFORMATION

None.

### FIELD OF THE INVENTION

The present invention relates to stabilizing compositions and methods and kits for chemiluminescent assays that use or contain said stabilizing compositions for the detection or quantification of at least one analyte of interest in a test sample.

### BACKGROUND OF THE INVENTION

Many assay systems for determining the concentration of one or more analytes of interest in a test sample are known in the art. Such assays find a wide scope of application ranging from the determination of toxic substances in industrial wastes to the determination of potential contaminants in food supplies to the identification or quantification of certain specific analytes in biological fluids, such as blood, serum, plasma, urine and the like. Moreover, many different types assay systems are known and many different types of signal labels can be used to identify one or more analytes of interest in such a test sample. Examples of some of the signal labels that are commonly used include, radioactive labels, enzyme labels, fluorescent labels and chemiluminescent labels.

Assays employing chemiluminescent labels are well known and have a number of significant advantages over other commonly used signal labels (such as radioactive, enzymes, etc.), such as a relatively high sensitivity, low cost, extended linear range, relatively simple signal measuring equipment and the lack of use of radioactive isotopes. Despite these advantages, the use of chemiluminescent assays has not been without problems. Specifically, the chemiluminescent signal is initiated via an oxidative chemical reaction. For example, the chemiluminescent signal from acridinium-9-carboxamides, acridium-9-carboxylate aryl esters, luminol, and isoluminol labels, is initiated by the reaction of the label with hydrogen peroxide in the presence of base and/or catalyst.

Adventitious oxidants, namely, those not specifically added to initiate the chemiluminescent reaction, which may be present in, or generated in, materials used in methods and kits for the detection or quantification of at least one analyte of interest in a test sample, have a de-stabilizing effect on chemiluminescent labels. Additionally, methods and kits for the detection or quantification of at least one analyte of interest in a test sample, use various proteins that are likewise de-stabilized by adventitious oxidants. For example, proteins such as enzymes, immunoglobulins and albumins contain oxidation-sensitive amino acid residues, that when oxidized, de-stabilize the native conformation of the protein thereby resulting in diminished or otherwise altered biological activity. Such oxidation-sensitive amino acid residues include cysteine, methionine tryptophan and tyrosine. The net result of de-stabilization of the chemiluminescent label or protein contained in an assay is a fluctuation in the detection signal that increases imprecision in the detection or quantification of at least one analyte of interest in a test sample. Moreover, efforts to remove the adventitious oxidant that may be initially present in the material used in the methods and kits may be rendered pointless due to the continuing generation of the adventitious oxidant over the time between preparation of the methods and kits and the detection or quantification of at least one analyte of interest in a test sample. Therefore, there is a need in the art for stabilizing compositions for use in chemiluminescent assay methods and contained in kits for the detection or quantification of at least one analyte of interest in a test sample.

### SUMMARY OF THE INVENTION

In one embodiment, the present invention relates to a stabilizing composition for use in a chemiluminescent assay for the detection or quantification of at least one analyte of interest in a test sample. Specifically, the stabilizing composition comprises at least one reagent suitable for use with an acridinium compound in an chemiluminescent assay and an effective amount of at least one hydrogen peroxide consuming agent. The hydrogen peroxide consuming agent stabilizes the composition against oxidative degradation by hydrogen peroxide.

The acridinium compound used in conjunction with the stabilizing composition is can be a acridinium-9-carboxamide. More specifically, the acridinium-9-carboxamide has a structure according to formula I:

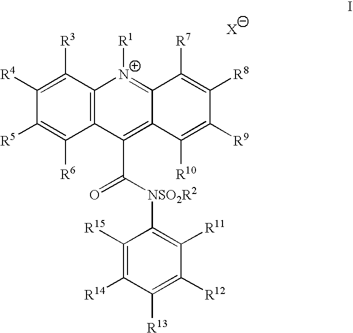

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Alternatively, the acridinium compound used in conjunction with the stabilizing composition is an acridinium-9-carboxylate aryl ester. The acridinium-9-carboxylate aryl ester has a structure of formula II:

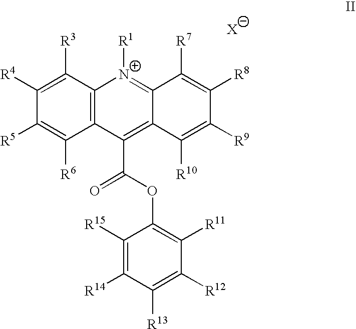

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

The reagent suitable for use with an acridinium compound and contained in the stabilizing composition of the present invention is selected from the group consisting of: a pre-treatment reagent, a detection reagent, a buffer, a diluent, a calibrator, a control or any combinations thereof.

The hydrogen peroxide consuming agent contained in the stabilizing composition of the present invention is selected from the group consisting of: an enzyme, a transition metal complex, a reducing agent and any combinations thereof. If the hydrogen peroxide consuming agent is an enzyme, the enzyme is selected from the group consisting of a haloperoxidase and a catalase. Examples of such an enzyme are myeloperoxidases, eosinophil peroxidases and lactoperoxidases. If the hydrogen peroxide consuming agent is a transition metal complex, the transition metal complex is an organometallic complex of vanadium, chromium, manganese, iron, cobalt, nickel or copper. If the hydrogen peroxide consuming agent is a reducing agent, the reducing agent is a reducing substance. Examples of reducing substances are bisulfites and ascorbic acid.

In another embodiment, the present invention relates to a method for detecting or quantifying at least one analyte of interest in a test sample. The method comprises the steps of:

a) adding an acridinium compound to a test sample;

b) adding the stabilizing composition of the present invention described above to the test sample either before or after the addition of the acridinium compound to the test sample;

c) adding hydrogen peroxide to the test sample;

d) adding a basic solution to the test sample to generate a light signal; and

e) measuring the light generated to detect or quantify the analyte of interest in the test sample.

In the above method, the acridinium compound is an acridinium-9-carboxamide, an acridinium-9-carboxylate aryl ester or any combinations thereof. The acridinium-9-carboxamide has a structure according to formula I:

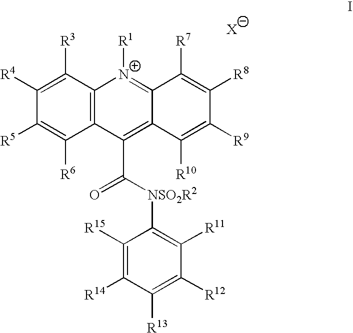

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

The acridinium-9-carboxylate aryl ester has a structure according to formula II:

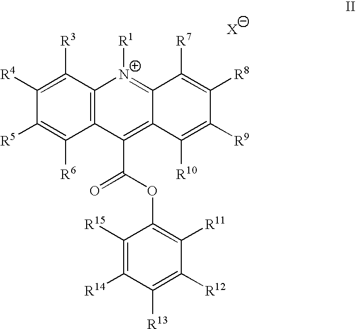

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

In the above method, the sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

The above method can further comprise quantifying the amount of the hydrogen peroxide in the test sample by relating the amount of light generated in the test sample by comparison to a standard curve for said analyte. Specifically, the standard curve is generated from solutions of an analyte of a known concentration.

In yet another embodiment, the present invention relates to a kit for the detection or quantification of at least one analyte of interest in a test sample. The kit can comprise the above-described stabilizing composition of the present invention. Moreover, the kit can further comprise at least one acridinium detection reagent (namely, an acridinium compound), at least one basic solution and instructions for the detection or quantification of at least one analyte of interest in a test sample, or any combinations thereof.

If the kit contains at least one acridinium detection reagent, the acridinium detection reagent may comprise at least one acridinium-9-carboxamide, at least one acridinium-9-carboxylate aryl ester or any combinations thereof.

If the kit contains at least one acridinium-9-carboxamide, the acridinium-9-carboxamide can have a structure according to formula I:

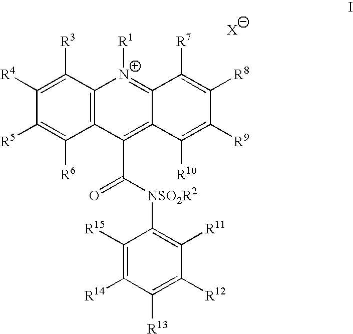

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Alternatively, if the kit contains at least one acridinium-9-carboxylate aryl ester, the acridinium-9-carboxylate aryl ester can have a structure according to formula II:

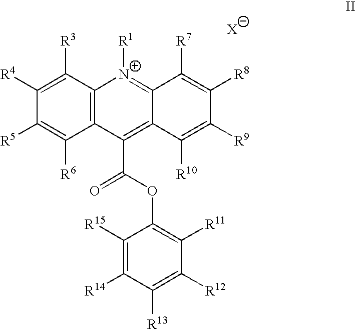

wherein R¹ is an alkyl, alkenyl, alkynyl, aryl or aralkyl, sulfoalkyl, carboxyalkyl and oxoalkyl; and

R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

### BRIEF DESCRIPTION OF THE FIGURES

FIG. 1 shows the myeloperoxidase (MPO) dose response at 150 μM hydrogen peroxide pursuant to Example 1.

FIG. 2 shows the MPO dose response at 75 μM hydrogen peroxide pursuant to Example 1.

FIG. 3 shows the MPO dose response at 50 μM hydrogen peroxide pursuant to Example 1.

FIG. 4 shows the MPO dose response at 30 μM hydrogen peroxide pursuant to Example 1.

FIG. 5 shows the MPO dose response at 20 μM hydrogen peroxide pursuant to Example 1.

FIG. 6 shows the MPO dose response at 10 μM hydrogen peroxide pursuant to Example 1.

FIG. 7 shows the MPO dose response at 5 μM hydrogen peroxide pursuant to Example 1.

FIG. 8 shows the MPO dose response at 0 μM hydrogen peroxide pursuant to Example 1.

FIG. 9 shows the hydrogen peroxide reduction with the addition MPO to a test sample pursuant to Example 1.

FIGS. 10A-C show the hydrogen peroxide reduction with the addition of a catalase to a test sample pursuant to Example 3.

FIG. 11 shows the structure of 9-[[(3-Carboxypropyl)[(4-methylphenyl)sulfonyl]amino]-carbonyl]-10-(3-sulfopropyl)acridinium inner salt.

### DETAILED DESCRIPTION OF THE INVENTION

The present invention provides a stabilizing composition for use in chemiluminescent assays for the detection or quantification of at least one analyte of interest in a test sample. Specifically, the stabilizing composition comprises at least one reagent for use with an acridinium compound and an effective amount of at least one hydrogen peroxide consuming agent. The hydrogen peroxide consuming agent stabilizes the composition against oxidative degradation by hydrogen peroxide. The stabilizing composition of the present invention can be used in one or more steps of a method or as a component of a kit in the detection or quantification of at least one analyte of interest in a test sample. The stabilizing composition can be used in a method as part of a sample pre-treatment step or it can be included as a component in a kit, such as a detection reagent, a buffer, a diluent, a calibrator or a control. Preferably, the stabilizing composition is present as a step or as a component of a method or kit for the detection or quantification of at least one analyte of interest in a test sample, which contains a chemiluminescent acridinium compound. More preferably, the stabilizing composition is present as a step or as a component of any step or component of a method or kit for the detection or quantification of at least one analyte of interest in a test sample, which contains a chemiluminescent acridinium compound and a protein.

A. Definitions

As used herein, the term “acyl” refers to a —C(O)Rₐ group where Rₐ is hydrogen, alkyl, cycloalkyl, cycloalkylalkyl, phenyl or phenylalkyl. Representative examples of acyl include, but are not limited to, formyl, acetyl, cylcohexylcarbonyl, cyclohexylmethylcarbonyl, benzoyl, benzylcarbonyl and the like.

As used herein, the term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “alkyl radical” means any of a series of univalent groups of the general formula CₙH₂ₙ₊₁ derived from straight or branched chain hydrocarbons.

As used herein, the term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, the term “amido” refers to an amino group attached to the parent molecular moiety through a carbonyl group (wherein the term “carbonyl group” refers to a —C(O)— group).

As used herein, the term “amino” means —NR_{b}R_{c}, wherein R_{b} and R_{c} are independently selected from the group consisting of hydrogen, alkyl and alkylcarbonyl.

As used herein, the term “anion” refers to an anion of an inorganic or organic acid, such as, but not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid and fluorosulfonic acid and any combinations thereof.

As used herein, the term “aralkyl” means an aryl group appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

As used herein, the term “aryl” means a phenyl group, or a bicyclic or tricyclic fused ring system wherein one or more of the fused rings is a phenyl group. Bicyclic fused ring systems are exemplified by a phenyl group fused to a cycloalkenyl group, a cycloalkyl group, or another phenyl group. Tricyclic fused ring systems are exemplified by a bicyclic fused ring system fused to a cycloalkenyl group, a cycloalkyl group, as defined herein or another phenyl group. Representative examples of aryl include, but are not limited to, anthracenyl, azulenyl, fluorenyl, indanyl, indenyl, naphthyl, phenyl, and tetrahydronaphthyl. The aryl groups of the present invention can be optionally substituted with one-, two, three, four, or five substituents independently selected from the group consisting of alkoxy, alkyl, carboxyl, halo, and hydroxyl.

As used herein, the term “carboxy” or “carboxyl” refers to —CO₂H or —CO₂⁻.

As used herein, the term “carboxyalkyl” refers to a —(CH₂)ₙCO₂H or —(CH₂)ₙCO₂⁻group where n is from 1 to 10.

As used herein, the term “cyano” means a —CN group.

As used herein, the term “cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine-to ten-membered ring has one to four double bonds. Representative examples of cycloalkenyl groups include cyclohexenyl, octahydronaphthalenyl, norbornylenyl, and the like. The cycloalkenyl groups can be optionally substituted with one, two, three, four, or five substituents independently selected from the group consisting of alkoxy, alkyl, carboxyl, halo, and hydroxyl.

As used herein, the term “cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present invention can be optionally substituted with one, two, three, four, or five substituents independently selected from the group consisting of alkoxy, alkyl, carboxyl, halo, and hydroxyl.

As used herein, the term “cycloalkylalkyl” means a —R_{d}Rₑ group where R_{d} is an alkylene group and Rₑ is cycloalkyl group. A representative example of a cycloalkylalkyl group is cyclohexylmethyl and the like.

As used herein, the term “halogen” means a —Cl, —Br, —I or —F; the term “halide” means a binary compound, of which one part is a halogen atom and the other part is an element or radical that is less electronegative than the halogen, e.g., an alkyl radical.

As used herein, the term “hydrogen peroxide consuming agent” refers to any composition, reagent or molecule that is capable of reducing or consuming hydrogen peroxide. Examples of hydrogen peroxide consuming agents include, but are not limited to, an enzyme, a transition metal complex, a reducing agent and any combinations thereof. Examples of enzymes which can be used as hydrogen peroxide consuming agents include, but are not limited to, haloperoxidases and catalases. Examples of haloperoxidases and catalases that are suitable for use in the present invention are those described in Messerschmidt, A., Huber, R., Poulus, T., and Wieghardt, K., (Eds.), Handbook of Metalloproteins (2004), Wiley; and Sykes, A., and Mauk, G., Heme-Fe Proteins Vol. 51, Academic Press (2000), the contents of which are herein incorporated by reference in their entirety. For example, myeloperoxidases, eosinophil peroxidases and lactoperoxidases are examples of enzymes that can be used as hydrogen peroxide consuming agents. A transition metal complex that can be used as a hydrogen peroxide consuming agent is an organometallic complex of vanadium, chromium, manganese, iron, cobalt, nickel or copper. Preferably, the transition metal complex is an iron-containing complex. More preferably, the transition metal complex is an iron complex with triethylenetetramine as described in Wang, et al. (See, Wang, J. H., Acc. Chem. Res. 3, 90-97 (1970); Jarnagin, R. C., and Wang, J. H., J. Am. Chem. Soc. 80, 6477-6481 (1958); Wang, J. H., J. Am. Chem. Soc. 77, 4715-4719 (1955); Wang, J. H., J. Am. Chem. Soc. 77, 822-823 (1955)). The reducing agent that is used is a reducing substance. Specifically, the reducing substances are selected from the group consisting of: a bisulfite and ascorbic acid.

As used herein, the term “hydroxyl” means an —OH group.

As used herein, the term “nitro” means a —NO₂ group.

As used herein, the term “oxoalkyl” refers to —(CH₂)ₙC(O)Rₐ, where Rₐ is hydrogen, alkyl, cycloalkyl, cycloalkylalkyl, phenyl or phenylalkyl and where n is from 1 to 10.

As used herein, the term “phenylalkyl” means an alkyl group which is substituted by a phenyl group.

As used herein, the term “sulfo” means a —SO₃H group.

As used herein, the term “sulfoalkyl” refers to a —(CH₂)ₙSO₃H or —(CH₂)ₙSO₃⁻ group where n is from 1 to 10.

As used herein, the term “test sample” generally refers to a biological material being tested for and/or suspected of containing an “analyte” or “analyte of interest”, as used interchangeably herein, generally refers to a substance to be detected. Analytes may include inorganic substances, including, but not limited to, hydrogen peroxide and sulfite. Analytes include, but are not limited to, antigenic substances, haptens, antibodies, and combinations thereof. Analytes include, but are not limited to, toxins, organic compounds, DNA, RNA, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Examples of some analytes include, but are not limited to, brain natriuretic peptide (BNP) 1-32; NT-proBNP; proBNP; preproBNP; troponin I; troponin T; troponin C; human neutrophil gelatinase-associated lipocalin (hNGAL); tacrolimus; sirolimus, cyclosporine; ferritin; creatinine kinase MB (CK-MB); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HbsAg); antibodies to hepatitis B core antigen, such as anti-hepatitis B core antigen IgG and IgM (Anti-HBC); human immune deficiency virus (HIV); human T-cell leukemia virus (HTLV); hepatitis B e antigen (HbeAg); antibodies to hepatitis B e antigen (Anti-Hbe); influenza virus; thyroid stimulating hormone (TSH); thyroxine (T4); total triiodothyronine (Total T3); free triiodothyronine (Free T3); carcinoembryonic antigen (CEA); lipoproteins, cholesterol, and triglycerides; galactose, glucose, LDL, HDL, choline, lactic acid, uric acid, phosphatidylcholine, acetylcholine, phosphocholine, CDP-choline, lysophosphatidylcholine, phospholipase A2, phosholipase D, lysophosholipase D and sphingomyelin and alpha fetoprotein (AFP). Drugs of abuse and controlled substances include, but are not limited to, amphetamine; methamphetamine; barbiturates, such as amobarbital, secobarbital, pentobarbital, phenobarbital, and barbital; benzodiazepines, such as propoxy and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyphene or any combinations thereof. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

As used in this specification and the appended claims, the singular forms “a”, “an”, and “the” include plural references unless the context clearly dictates otherwise. Thus, for example, a reference to “the method” includes one or more methods, and/or steps of the type described herein and/or which will become apparent to those persons skilled in the art upon reading this disclosure and so forth.

B. Stabilizing Compositions and Methods for Detecting or Quantifying at Least One Analyte of Interest in A Chemiluminescent Assay

In one embodiment, the present invention relates to a stabilizing composition that can be used in chemiluminescent assays for the detection or quantification of at least one analyte of interest in a test sample. The composition of present invention is considered to be “stabilizing” in the sense that it is protects the assay components against oxidative degradation from adventitious hydrogen peroxide. While not wishing to be bound by any theory, the inventors believe that such stabilizing effect is first a chemical stabilization, and second, a stabilization in the detection signal that increases the precision in the detection or quantification of at least one analyte of interest in a test sample.

The stabilizing composition of the present invention comprises at least one reagent suitable for use with an acridinium compound in a chemiluminescent assay. Such a reagent can be a reagent used in a pre-treatment step (a pre-treatment reagent), a detection reagent, a buffer, a diluent, a calibrator, control or any combinations thereof. An example of a pre-treatment reagent that can be used is a whole blood precipitation reagent, such as the whole blood precipitation reagent that is used in connection with the Abbott Laboratories ARCHITECT® iSystem sirolimus and tacrolimus assays (commercially available from Abbott Laboratories under list number 1L76 (sirolimus) and 1L77 (tacrolimus)). Generally, this whole blood precipitation reagent contains ZnSO₄, DMSO and ethylene glycol. Examples of calibrators and controls that can be used are those commercially available from Abbott Laboratories as part of its ARCHITECT® iSystem Assay Kit. Specifically, the commercially available assay kits for each of the assays described below in Table A contain at least one calibrator and at least one control that can be used as a reagent in the present invention.

TABLE A

| Abbott Laboratories | |
|---|---|
| Assay Name | List Number |
| AFP | 7K67 |
| AFP 2 | 7K67 |
| Anti-HBc | 7C17 |
| Anti-HBc IgM | 6C33 |
| Anti-HBe | 6C34 |
| Anti-HBs | 7C18 |
| Anti-HCV | 6C37 |
| | 1L79 |
| Anti-Tg | 2K46 |
| Anti-TPO | 2K47 |
| AUSAB | 1L82 |
| B12 | 6C09 |
| B-hCG STAT | 7K78 |
| B-hCG | 6C21 |
| BNP | 8K28 |
| BNP STAT | |
| CA 125 II | 2K45 |
| CA 15-3 | 2K44 |
| CA 19-9_{XR} | 2K91 |
| CEA | 7K68 |
| | 6C02 |
| CK-MB, STAT | 2K42 |
| CMV IgG | 6C15 |
| CMV IgM | 6C16 |
| CORE-M | 6L23 |
| Cortisol | 8D15 |
| DHEA-S | 8K27 |
| Estradiol | 7K72 |
| | 2K25 |
| Ferritin | 6C11 |
| | 7K59 |
| | 6C11 |
| Folate RBC | 7K60 |
| Folate | |
| Free PSA | 7K71 |
| | 6C07 |
| Free T₃ | 7K63 |
| | 6C48 |
| Free T₄ | 7K65 |
| | 7G96 |
| | 6C50 |
| FSH | 7K75 |
| | 6C24 |
| HAVAb-IgG | 6C29 |
| HAVAb-IgM | 6C30 |
| HAVAB-M | 6L21 |
| HBeAg | 6C32 |
| HBsAg | 1L80 |
| HBsAg Confirmatory | 1L81 |
| HBsAg | 3M61 |
| | 6C36 |
| HBsAg Confirmatory | 9C94 |
| HIV Ag/Ab Combo | 4J27 |
| Insulin | 8K41 |
| Intact PTH | 8K25 |
| Intact PTH STAT | |
| LH | 7K74 |
| | 6C25 |
| Myoglobin, STAT | 2K43 |
| iPhenytoin | 1P34 |
| Progesterone | 7K77 |
| | 6C26 |
| Prolactin | 7K76 |
| | 6C27 |
| Rubella IgG | 6C17 |
| Rubella IgM | 6C18 |
| SCC | 8D18 |
| SHBG | 8K26 |
| Sirolimus | 1L76 |
| Syphilis TP | 8D06 |
| Tacrolimus | 1L77 |
| Testosterone | 7K73 |
| | 6C28 |
| iTheophylline | 1P29 |
| Total PSA | 7K70 |
| | 6C06 |
| Total T₃ | 7K64 |
| | 6C51 |
| Total T₄ | 7K66 |
| | 6C49 |
| Troponin-I, STAT | 2K41 |
| Toxo IgG | 6C19 |
| TSH | 7K62 |
| | 6C52 |
| T-Uptake | 2K48 |
| iVancomycin | 1P30 |

Examples of buffers that can be used include, but are not limited to, acetate buffers (such as sodium acetate/acetic acid, potassium acetate/acetic acid), citrate buffers (such as sodium citrate/citric acid, potassium citrate/citric acid), phosphate buffers (such as mono-/di-sodium phosphate) or combinations thereof. Any diluent can be used in the composition of the present invention. For example, a diluent that can be used can be made using techniques known in the art or can be a purchased from a commercially available source. Combinations of both custom made diluents and commercially purchased diluents are also contemplated within the scope of the present invention. Additionally, the composition of the present invention also contains an effective amount of at least one hydrogen peroxide consuming agent. The at least one hydrogen peroxide consuming agent stabilizes the composition against oxidative degradation by hydrogen peroxide. As used herein, the phrase “effective amount” of the hydrogen peroxide consuming agent refers to the minimum amount necessary to reduce the adventitious hydrogen peroxide concentration by at least 50% in the time period between preparation of the composition and performing the chemiluminescent assay for at least one analyte of interest in a test sample. The “effective amount” may be calculated based on the activity of the agent, which is usually given in units of “moles of hydrogen peroxide consumed” per “weight of agent” per “time period”, the “turnover number” which is the number of moles of hydrogen peroxide that a mole of the agent can consume before becoming inactivated, the initial concentration of the adventitious hydrogen peroxide and the rate of formation of adventitious hydrogen peroxide. The stabilizing composition of the present invention can be in any form, but is preferably in the form of a liquid.

In addition to the at least one reagent and at least one hydrogen peroxide consuming agent, the composition can contain at least one biocide, preservative or any combination of at least one biocide and at least one preservative. As used herein, the term “biocide” refers to a substance that can be used to kill a variety of different organisms. Suitable biocides or preservatives for use in the composition can be determined using routine techniques by those skilled in the art. Examples of suitable biocides and/or preservatives that can be used in the present invention, include, but are not limited to ProClin® 300 (Sigma-Aldrich, St. Louis, Mo.) (The active ingredients of ProClin®300 are 5-chloro-2-methyl-4-isothiazolin-3-one and 2-methyl-4-isothiazolin-3-one and sodium azide. ProClin® 300 also contains a number of inert ingredients such as a modified glycol and alkyl carboxylate.). The amount of biocide and/or preservative in the composition can be from about 0.001 mg/mL to about 50 mg/mL, preferably in the amount of from about 0.1 mg/mL to about 10 mg/mL.

Furthermore, the composition can also comprise at least one acid, at least one base or any combinations thereof. It is preferred that the buffer have a good buffering capacity at the desired pH so that it can stabilize the pH of the composition. Examples of acids that can be used include, but are not limited to, acetic acid, diethylenetriaminepentaacetic acid (DTPA), hydrochloric acid (HCl) or combinations thereof. The amount of acid in the composition can be from about 1 mM to about 500 mM, preferably in the amount of from about 5 to about 200 mM. An example of a base that can be used includes, but is not limited to, sodium hydroxide (NaOH). The amount of base in the composition can be from about 0.01 mM to about 50 mM, preferably in the amount of from about 1.0 mM to about 10 mM.

The composition can also comprise sodium chloride (NaCl) and/or at least one detergent, such as Tween® (Any type of Tween® can be used, including, but not limited to, Tween® 20, Tween® 40, Tween® 60, which are commercially available from Sigma-Aldrich, St. Louis, Mo.). The amount of NaCl in the diluent can be from about 25 mM to about 500 mM, preferably in the amount of from about 100 mM to about 400 mM. The amount of detergent in the diluent can be from about 0.01 mg/mL to about 10 mg/mL, preferably in the amount of from about 0.1 mg/mL to about 3.0 mg/mL.

As alluded to above, the stabilizing composition of the present invention can be used with an acridinium compound in a chemiluminescent assay. Preferably, the acridinium compound is an acridinium-9-carboxamide. Specifically, the acridinium-9-carboxamide has a structure according to formula I:

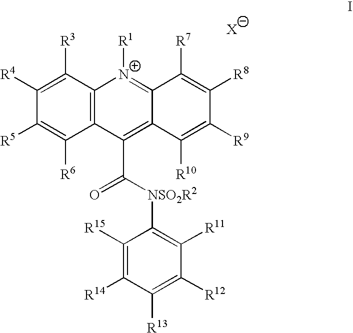

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl;

and further wherein any of the alkyl, alkenyl, alkynyl, aryl or aralkyl may contain one or more heteroatoms; and

optionally, if present, X^{⊖} is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524 and 5,783,699 (each incorporated herein by reference in their entireties for their teachings regarding same).

Alternatively, the acridinium compound can be an acridinium-9-carboxylate aryl ester; the acridinium-9-carboxylate aryl ester can have a structure according to formula II:

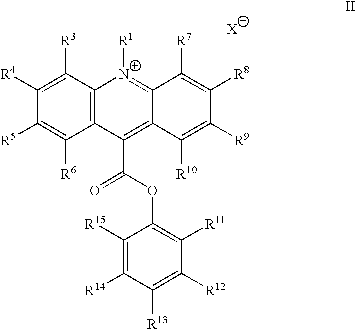

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Examples of acridinium-9-carboxylate aryl esters having the above formula II that can be used in the present invention include, but are not limited to, 10-methyl-9-(phenoxycarbonyl)acridinium fluorosulfonate (available from Cayman Chemical, Ann Arbor, Mich.). Methods for preparing acridinium 9-carboxylate aryl esters are described in McCapra, F., et al., Photochem. Photobiol., 4, 1111-21 (1965); Razavi, Z et al., Luminescence, 15:245-249 (2000); Razavi, Z et al., Luminescence, 15:239-244 (2000); and U.S. Pat. No. 5,241,070 (each incorporated herein by reference in their entireties for their teachings regarding same).

In a second embodiment, the present invention relates to a method for detecting or quantifying at least one analyte of interest from a subject. A subject from which a test sample can be obtained is any vertebrate. Preferably, the vertebrate is a mammal. Examples of mammals include, but are not limited to, dogs, cats, rabbits, mice, rats, goats, sheep, cows, pigs, horses, non-human primates and humans. The test sample can be obtained from the subject using routine techniques known to those skilled in the art.

After the test sample containing at least one analyte of interest is obtained from a subject, the concentration of the analyte is determined. Specifically, at least one acridinium compound is added to the test sample. The acridinium compound can be an acridinium-9-carboxamide, acridinium-9-carboxylate aryl ester or any combinations thereof. Acridinium-9-carboxamides that can be used in this method are those having a structure according to formula I shown below:

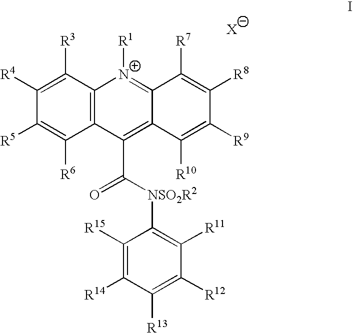

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl;

and further wherein any of the alkyl, alkenyl, alkynyl, aryl or aralkyl may contain one or more heteroatoms; and

optionally, if present, X^{⊖} is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524, and 5,783,699 (each incorporated herein by reference in their entireties for their teachings regarding same).

Acridinium-9-carboxylate aryl esters that can be used in this method are those having a structure according to formula II shown below:

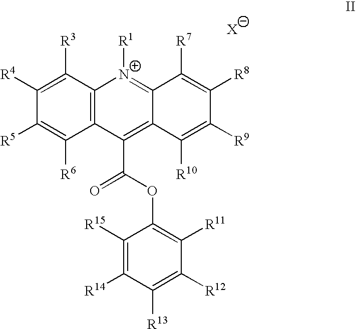

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Methods for preparing acridinium 9-carboxylate aryl esters are described in McCapra, F., et al., Photochem. Photobiol., 4, 1111-21 (1965); Razavi, Z et al., Luminescence, 15:245-249 (2000); Razavi, Z et al., Luminescence, 15:239-244 (2000); and U.S. Pat. No. 5,241,070 (each incorporated herein by reference in their entireties for their teachings regarding same).

The stabilizing composition can be added to the test sample prior to the addition of the acridinium compound(s) (such as a pre-treatment step) or it may be added after the addition of the acridinium compound(s). In such an instance, the stabilizing composition can be added sequentially or simultaneously with the acridinium compound(s).

As alluded to above, the timing and order in which the acridinium compound(s) is supplied to the test sample is not critical provided that it is added prior to the addition of at least one basic solution, which will be discussed in more detail below.

After the addition of the stabilizing composition of the present invention and the acridinium compound(s) to the test sample, at least one basic solution is added to the test sample in order to generate a detectable signal, namely, a first chemiluminescent signal. The basic solution is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the test sample depends on the concentration of the basic solution used in the assay. Based on the concentration of the basic solution used, one skilled in the art could easily determine the amount of basic solution to be used in the method. Chemiluminescent signals generated can be detected using routine techniques known to those skilled in the art.

Thus, the chemiluminescent signal generated after the addition of a basic solution, indicates the presence of the analyte of interest. The amount of the analyte in the test sample can be quantified based on the intensity of the first signal generated. Specifically, the amount of the analyte contained in a test sample is proportional to the first signal generated. Specifically, the amount of the analyte of interest present can be quantified based on comparing the amount of light generated to a standard curve for the analyte or by comparison to a reference standard. The standard curve can be generated using serial dilutions or solutions of analyte of interest of known concentration, by mass spectroscopy, gravimetrically and by other techniques known in the art.

C. Chemiluminescent Assay Kit Containing a Stabilizing Composition for a use in a Chemiluminescent Assay

In another embodiment, the present invention relates to a kit containing a stabilizing composition for performing a chemiluminescent assay for detecting or determining at least one analyte in a test sample. In one aspect, the kit contains the stabilizing composition of the present invention described previously herein. In addition, the kit can also contain at least one acridinium compound. If the kit contains at least one acridinium compound, the acridinium compound may comprise at least one acridinium-9-carboxamide, at least one acridinium-9-carboxylate aryl ester or any combinations thereof. More specifically, the acridinium-9-carboxamide that can be used has the structure according to Formula I:

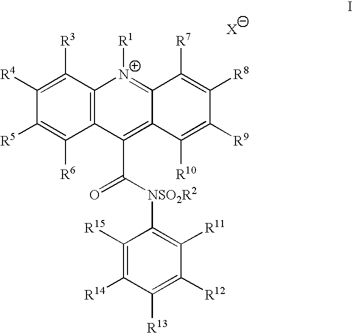

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

further wherein any of the alkyl, alkenyl, alkynyl, aryl or aralkyl may contain one or more heteroatoms; and

optionally, if present, X^{⊖} is an anion.

Additionally, the acridinium-9-carboxylate aryl ester that can be used has a structure according to formula II:

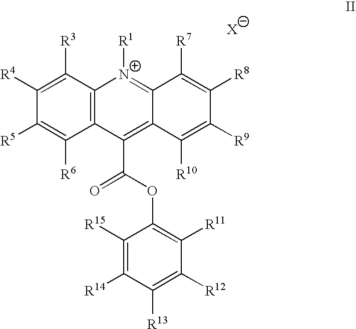

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Additionally, the kit can also contain at least one basic solution.

Also, the kit can also contain one or more instructions for detecting and quantifying at least one analyte in a test sample. The kit can also contain instructions for generating a standard curve for the purposes of quantifying at least one analyte in the test sample. Such instructions optionally can be in printed form or on CD, DVD, or other format of recorded media.

By way of example, and not of limitation, examples of the present invention shall now be provided.

### EXAMPLE 1

### Dose Response for Reducing Hydrogen Peroxide Concentration Using Myeloperoxidase

Standard Solutions of Hydrogen Peroxide.

Hydrogen peroxide (30% aqueous, ACS grade, JTBaker #2186-01) was diluted in reagent grade water to give solutions of 150, 75, 50, 30, 20, 10, 5 and 0 μM.

Standard Solutions of Myeloperoxidase.

Myeloperoxidase from human leukocytes (Sigma Catalog #M6908) was diluted in phosphate buffered saline (PBS, pH 7.2) containing methionine (1 mM) to give solutions of 2900, 1450, 725, 362.50, 181.25, 90.63, 45.31, 22.66, 11.33, and 0.00 ng/mL.

Chemiluminescent Detection Reagent.

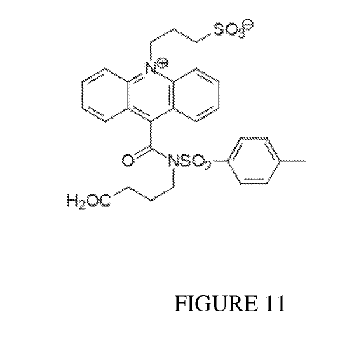

9-[[(3-Carboxypropyl)[(4-methylphenyl)sulfonyl]amino]-carbonyl]-10-(3-sulfopropyl)acridinium inner salt (See, FIG. 11) was dissolved in reagent grade water containing sodium cholate (0.1% wt/volume) to give a concentration of 250 nM.

Assay Protocol.

The standard hydrogen peroxide solutions (4 μL) and the standard myeloperoxidase solutions (20 μL) were arrayed in triplicate in a 96-well microplate. The plate incubated in a microplate luminometer (Mithras LB-940, BERTHOLD TECHNOLOGIES U.S.A. LLC, Oak Ridge, Tenn.) at 37° C. for 30 minutes. Well by well, the chemiluminescent detection reagent (40 μL) and aqueous sodium hydroxide (0.25 N, 100 μL) were sequentially added and the chemiluminescent signal recorded for 2 seconds.

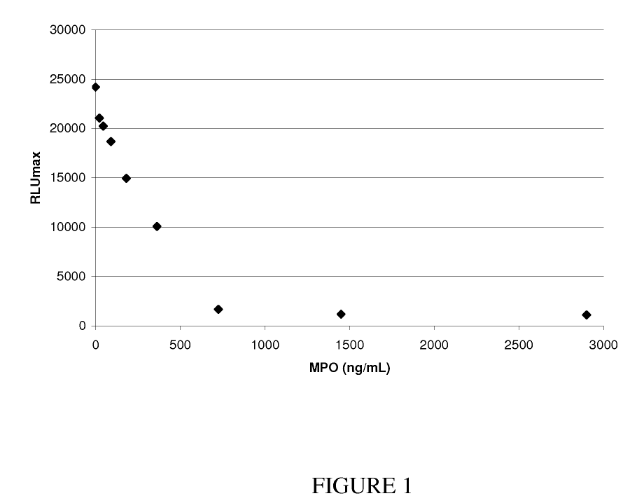

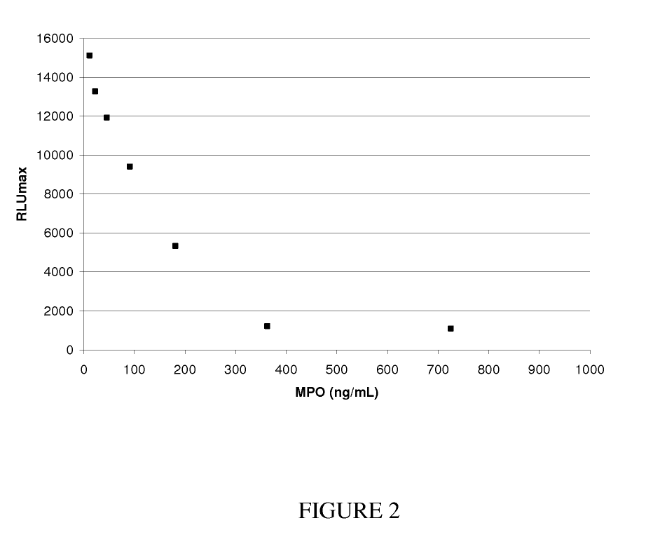

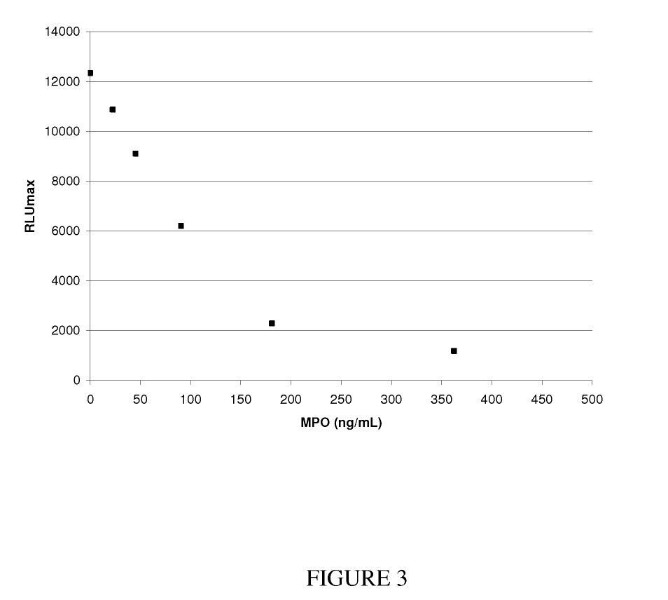

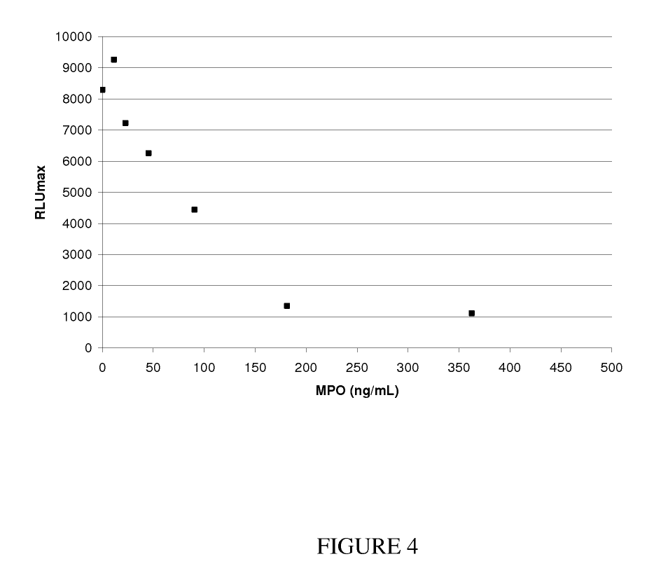

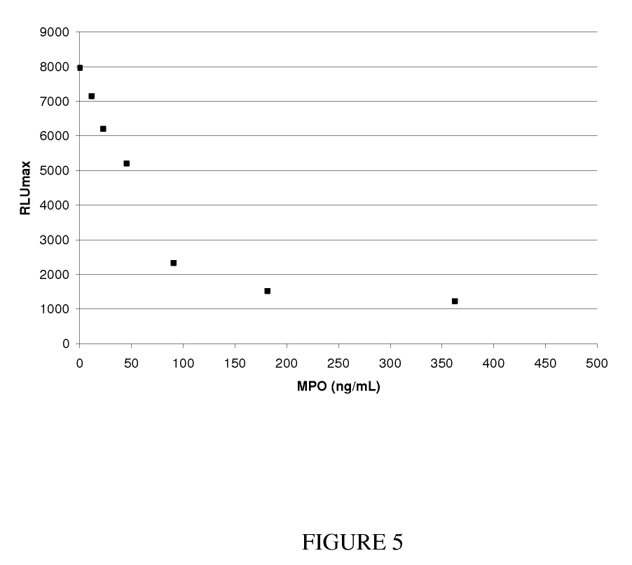

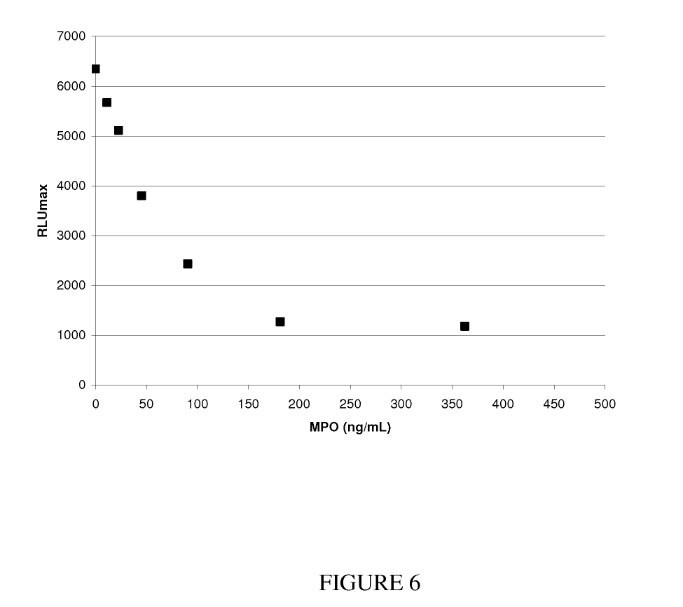

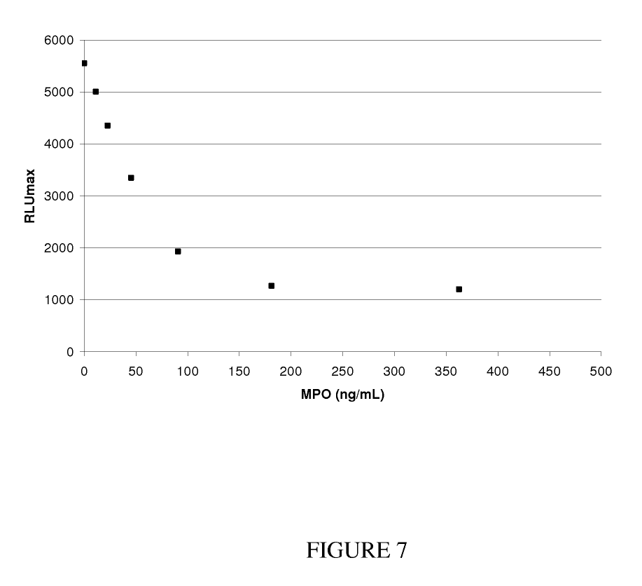

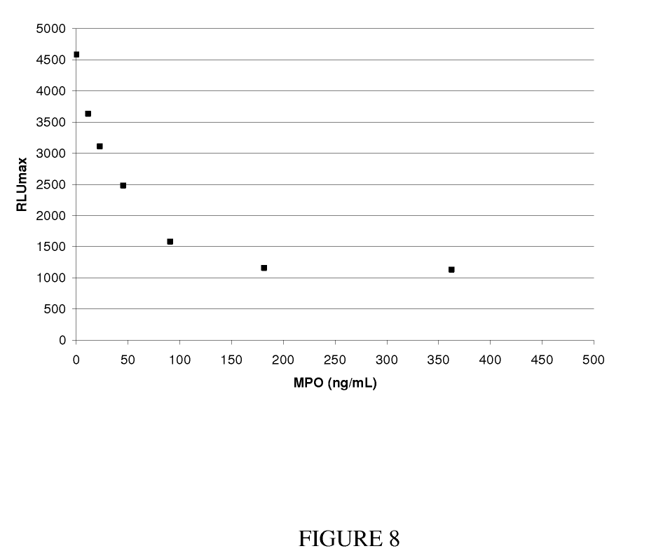

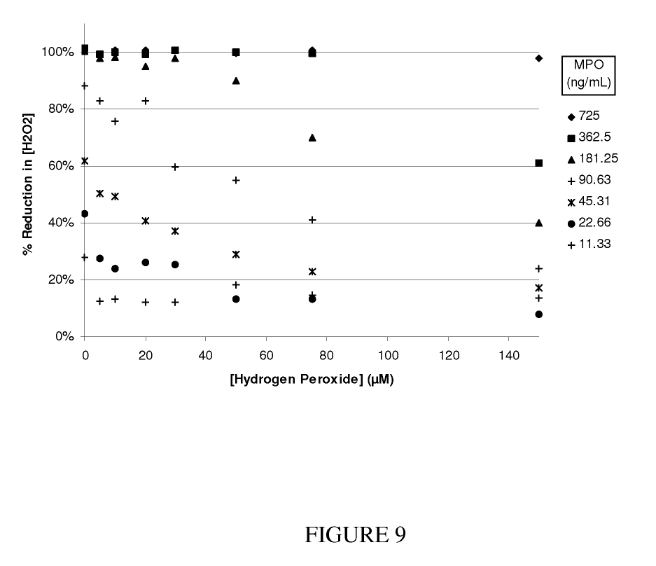

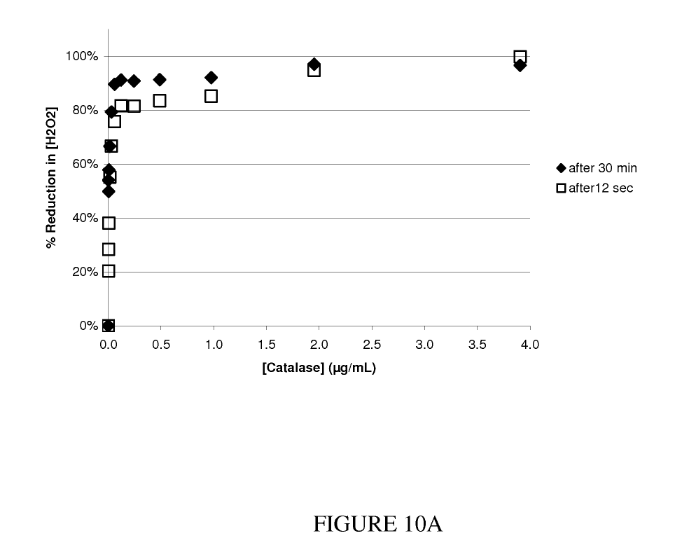

The resulting dose response (chemiluminescent signal/myeloperoxidase concentration) at each of the hydrogen peroxide concentrations tested is shown in FIGS. 1 to 8. Additionally, FIG. 8 shows that nascent hydrogen peroxide is present in the solvents used in the reagent preparation and that nascent peroxide can be reduce by the addition of MPO. FIG. 9 further illustrates the percent reduction of hydrogen peroxide over a range of concentrations at different MPO concentrations.

### EXAMPLE 2

### Reduction of Hydrogen Peroxide Using Catalase

Catalase Stock Solution.

Catalase (20 mg, Sigma Catalog #C-40) was dissolved in PBS (10 mL) to give a 2 mg/mL solution.

Chemiluminescent Detection Reagent.

9-[[(3-Carboxypropyl)[(4-methylphenyl)sulfonyl]amino]-carbonyl]-10-(3-sulfopropyl)acridinium inner salt (See, FIG. 11) was dissolved in reagent grade water containing sodium cholate (0.1% wt/volume) to give a concentration of 4 μM.

Assay Protocol.

The catalase stock solution was serial diluted (1:2) with pH 8 phosphate. The dilutions were arrayed in quadruplicate in a 96-well microplate. The final volume in each well was 40 μL. The plate was placed in a microplate luminometer (Mithras LB-940, BERTHOLD TECHNOLOGIES U.S.A. LLC, Oak Ridge, Tenn.) at 28° C. Well by well, the chemiluminescent detection reagent (40 μL) was dispensed, then 12 seconds later, aqueous sodium hydroxide (0.25 N, 100 μL) was dispensed and the chemiluminescent signal recorded for 2 seconds.

### EXAMPLE 3

### Effect of Delay Time on Reduction of Hydrogen Peroxide Using Catalase

The experiment of Example 2 was repeated using a delay time of 30 minutes instead of 12 seconds between the addition of the chemiluminescent reagent and the aqueous sodium hydroxide solution.

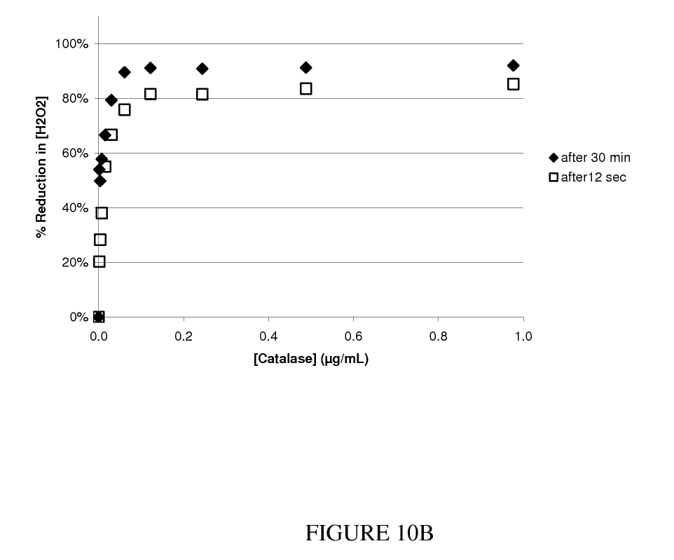

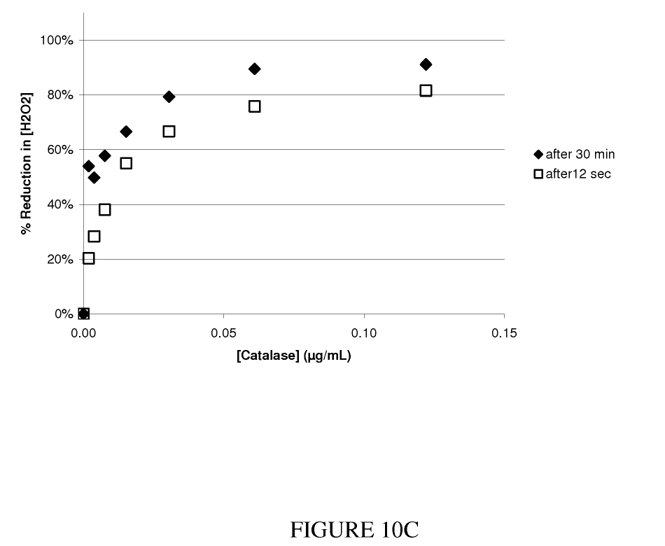

FIGS. 10A-C show that catalase reduces the nascent hydrogen peroxide in the reagent solutions at all concentrations tested. Further, also shown is that the effectiveness of the enzyme is both concentration and time dependent, e.g., at the lowest concentration tested (0.002 μg/mL) catalase had an effectiveness of 20% when measured after 12 seconds, but that effectiveness increased to 54% when measured 30 minutes later. Likewise, increasing the enzyme concentration to 3.9 μg/mL resulted in 100% effectiveness even only after 12 seconds.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A stabilizing composition for use in a chemiluminescent assay comprising: at least one reagent suitable for use with a detection agent and an effective amount of at least one hydrogen peroxide consuming agent, wherein said hydrogen peroxide consuming agent stabilizes the composition against oxidative degradation by hydrogen peroxide.

2. The composition of claim 1, wherein the reagent is selected from the group consisting of a pre-treatment reagent, a detection reagent, a buffer, a diluent, a calibrator, a control or any combinations thereof

3. The composition of claim 1, wherein the detection reagent is an acridinium compound.

4. The composition of claim 3, wherein the acridinium compound is an acridinium-9-carboxamide having a structure according to formula I: 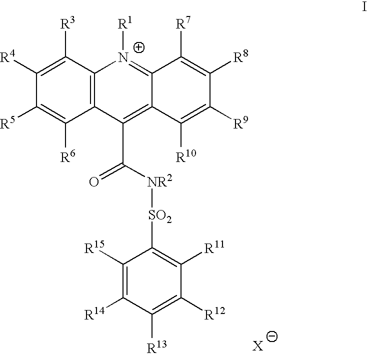
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

5. The composition of claim 3, wherein the acridinium compound is an acridinium-9-carboxylate aryl ester having a structure according to formula II: 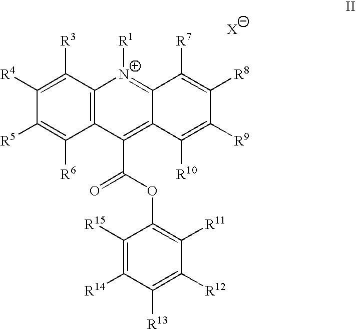
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

6. The composition of claim 1, wherein the hydrogen peroxide consuming agent is selected from the group consisting of: an enzyme, a transition metal complex, a reducing agent and combinations thereof.

7. The composition of claim 6, wherein the enzyme is selected from the group consisting of: a haloperoxidase and a catalase.

8. The composition of claim 7, wherein the enzyme is selected from the group consisting of: myeloperoxidase, eosinophil peroxidase, and lactoperoxidase.

9. The composition of claim 5, wherein the transition metal complex is an organometallic complex of vanadium, chromium, manganese, iron, cobalt, nickel or copper.

10. The composition of claim 6, wherein the reducing agent is a reducing substance.

11. The composition of claim 10, wherein the reducing substances is selected from the group consisting of: a bisulfite and ascorbic acid.

12. A method for detecting or quantifying at least one analyte of interest in a test sample, the method comprising the steps of:
a) adding an acridinium compound to a test sample;
b) adding the stabilizing composition of claim 1 to the test sample either before or after the addition of the acridinium compound to the test sample;
c) adding hydrogen peroxide to the test sample;
d) adding a basic solution to the test sample to generate a light signal; and
e) measuring the light generated to detect or quantify the analyte of interest in the test sample.

13. The method of claim 12, wherein the acridinium compound is an acridinium-9-carboxamide.

14. The method of claim 13, wherein the acridinium compound is an acridinium-9-carboxamide having a structure according to formula I: 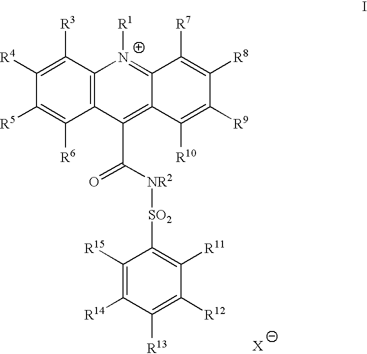
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

15. The method of claim 12, wherein the acridinium compound is an acridinium-9-carboxylate aryl ester having a structure according to formula II: 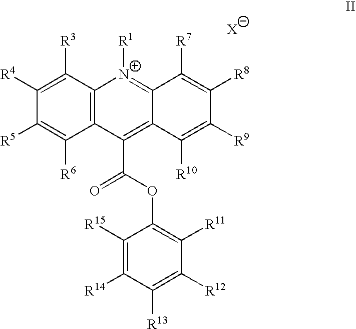
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

16. The method of claim 12, wherein the test sample is whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid or semen.

17. The method of claim 12, further comprising quantifying the amount of the analyte of interest in the test sample by relating the amount of light generated in the test sample by comparison to a standard curve for said analyte.

18. The method of claim 17, wherein the standard curve is generated from solutions of an analyte of a known concentration.

19. A kit for use in quantifying analyte of interest in a test sample, the kit comprising:
the stabilizing composition of claim 1.

20. The kit of claim 19, wherein said kit further comprises at least one acridinium compound, at least one basic solution, instructions for detecting analyte of interest in a test sample or any combinations thereof.

21. The kit of claim 19, wherein the acridinium compound is a acridinium-9-carboxamide having a structure according to formula I: 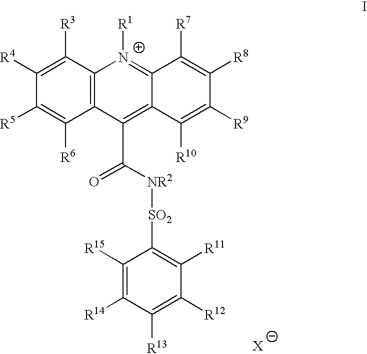
wherein R¹ and R² are each independently selected from the group consisting of: alkyl, alkenyl, alkynyl, aryl or aralkyl, sulfoalkyl, carboxyalkyl and oxoalkyl, and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

22. The kit of claim 19, wherein the acridinium compound is an acridinium-9-carboxylate aryl ester having a structure according to formula II: 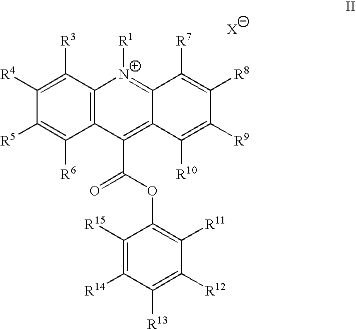
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.
